(19) United States
(12) Patent Application Publication
(10) Pub. No.: US 2006/0217579 A1
(43) Pub. Date: Sep. 28, 2006

(54) Selective hydrogenation process and catalyst therefor

(76) Inventor: Stephen Bailey, Cleveland (GB)

Correspondence Address: RATNERPRESTIA, P O BOX 980, VALLEY FORGE, PA, 19482-0980, US

(21) Appl. No.: 10/559,501

(22) PCT Filed: May 26, 2004
(86) PCT No.: PCT/GB04/02262

§ 371 Date: Mar. 24, 2006

(30) Foreign Application Priority Data
Jun. 4, 2003 (GB) 0312769.3

(51) Int. Cl. C07C 7/167; B01J 21/00; B01J 23/00

(52) U.S. Cl. 585/259; 502/302; 502/263; 502/303; 502/304

(57) ABSTRACT

A catalyst suitable for use in hydrogenation, especially the selective hydrogenation of acetylenic compounds to olefinic compounds, which comprises palladium supported upon an alumina support material characterised in that said catalyst further comprises a compound of a lanthanide.

The present invention relates to a process for selectively hydrogenating acetylene compounds in the presence of olefinic compounds. The invention also relates to a novel catalyst suitable for use in such a selective hydrogenation process.

The manufacture of unsaturated hydrocarbons usually involves cracking saturated and/or higher hydrocarbons and produces a crude product containing hydrocarbons that are more unsaturated than the desired product but which are very difficult to separate by fractionation. For example in the manufacture of ethylene, acetylene is a co-product. In polymer-grade ethylene specifications the acetylene content must be less than 10 ppm, typically 1-3 ppm max in the product ethylene, although some plants specify that the acetylene should be <0.5 ppm.

Because of the difficulty associated with separation of the olefin and acetylene co-products, it has long been the practice in industrial olefin manufacture to remove the acetylenic hydrocarbon product by hydrogenation of the triple bond to form an olefin. This approach carries the risk of hydrogenating the desired product olefin which forms a major component of the feed stream and also of over-hydrogenating the acetylene to produce saturated hydrocarbons. Therefore it is important to choose hydrogenation conditions which favour the hydrogenation of the acetylenic triple bonds but under which the olefinic double bonds, are not hydrogenated.

Two general types of gas-phase selective hydrogenation processes for purifying unsaturated hydrocarbons are used. “Front-end” hydrogenation involves passing the crude cracker product gas, from which steam and higher hydrocarbons (C₄+) have been removed, over a hydrogenation catalyst. The crude gas contains much more hydrogen than is required to effect hydrogenation of the acetylenic portion of the feed and therefore the potential for hydrogenation of the olefinic part of the gas stream is high. It is therefore important to choose an appropriately selective hydrogenation catalyst and control the conditions, especially temperature, to avoid unwanted hydrogenation of the olefins. In “tail-end” hydrogenation, the gaseous feed has already been separated from CO and H₂ and so the required amount of hydrogen for the hydrogenation reaction must be introduced into the reactor

In operating acetylene removal from olefin streams by front-end hydrogenation, where hydrogen is present in significant excess of the stoichiometric amount required to hydrogenate the acetylene, it is desirable to avoid hydrogenation of the olefin to a more saturated hydrocarbon. The hydrogenation process is sensitive to temperature, which varies according to the catalyst used. At relatively low temperatures, typically between about 55 and about 70° C., the acetylene is hydrogenated. The temperature at which at least about 99.9% of the acetylene has been hydrogenated is called the “clean-up” temperature (CUT). With a selective catalyst, olefin hydrogenation, which is highly exothermic, begins at a temperature of between 90 and 120° C., but the availability of hydrogen in the reactor can rapidly lead to thermal runaway and a consequent high level of unwanted olefin hydrogenation. The temperature at which the hydrogenation of olefin begins is called the “light-off temperature” (LOT). Therefore the window of operable temperature, i.e. the difference between the “light-off temperature” and the “clean-up temperature” should be as wide as possible so that a high conversion of acetylene can be achieved whilst avoiding the risk of olefin hydrogenation. This means that a successful catalyst for the selective hydrogenation of acetylenes in an olefin-rich feed gas should provide a high LOT-CUT. In tail-end hydrogenation processes, over-hydrogenation is less likely because there is less hydrogen in the gas stream than is the case for front-end hydrogenation. However selective catalysts are required in order to avoid the formation of hydrocarbons containing 4 or more carbon atoms, leading to oligomers and oil formation which reduces the activity of the catalyst.

Known catalysts for selective hydrogenation of acetylenes include Pd supported on alumina. U.S. Pat. No. 2,909,578 describes a catalyst comprising Pd supported on alumina, in which the Pd metal is about 0.00001-0.0014 percent of the total catalyst weight. U.S. Pat. No. 2,946,829 discloses selective hydrogenation catalysts in which Pd is supported on an alumina carrier having a pore volume of 0-0.4 cm³g⁻¹ at a threshold diameter of 800 Å or less.

U.S. Pat. No. 3,113,980 and U.S. Pat. No. 3,116,342 describe acetylene hydrogenation processes and catalysts comprising palladium supported on alumina whose pores have a mean radius not less than 100 Å and preferably not more than 1400 Å. The desired physical properties are obtained by heating an active alumina for at least 2 hours at a temperature in the range 800 to 1200° C. U.S. Pat. No. 4,126,645 describes a process of selective hydrogenation of highly unsaturated hydrocarbons in the presence of less unsaturated hydrocarbons characterised by the use of a catalyst which comprises palladium supported on particulate alumina having a surface area in the range 5 to 50 m²g⁻¹, a helium density of under 5 g cm⁻³, a mercury density of under 1.4 g cm⁻³ and a pore volume of at least 0.4 cm³g⁻¹, at least 0.1 cm³g⁻¹ of which is in pores of radius over 300 Å, the palladium being present mainly in the region of the catalyst particles not more than 150 microns beneath their geometric surface. Auxiliary materials such as zinc or vanadium oxide or Cu, Ag or Au metal may be present.

Whilst most supported Pd catalysts in use are of the “shell” type—i.e. having the Pd present only at or near the surface of the support particles, U.S. Pat. No. 3,549,720 describes the use of catalysts in which the Pd is uniformly distributed throughout the catalyst support, the alumina has a surface area above 80 m²g⁻¹ and the majority of the pores have diameters less than 800 Å. In U.S. Pat. No. 4,762,956, acetylene hydrogenation is carried out over a Pd on alumina catalyst in which the alumina has an average pore radius of 200-2000 Å, at least 80% of the pores having a pore radius within the range 100-3000 Å and which is formed by calcining the alumina support material at a temperature greater than 1150° C. but less than 1400° C.

Certain catalysts have been described in the art which contain certain promoters, usually one or more further metal species in addition to the Pd. For example, GB811820 describes acetylene hydrogenation using a catalyst containing 0.001 to 0.035% of palladium on activated alumina also containing 0.001 to 5% of copper, silver, gold, ruthenium, rhodium or iron as a promoter. EP-A-0124744 describes hydrogenation catalysts consisting of 0.1-60% by weight of a hydrogenating metal or of a hydrogenating metal compound of subgroup VIII of the periodic system of the elements on an inert support, containing 0.1-10% by weight of K₂O and, optionally, 0.001-10% by weight of an additive from the group comprising calcium, magnesium, barium, lithium, sodium, vanadium, silver, gold, copper and zinc, in each case based on the total weight of the catalyst, the K₂O doping being applied to a catalyst precursor consisting of the hydrogenating component, the support and, optionally, the additive. U.S. Pat. No. 3,821,323 describes the selective gas-phase hydrogenation of acetylene in an ethylene stream using a catalyst comprising palladium on silica-gel, additionally containing zinc. U.S. Pat. No. 4,001,344 describes catalysts comprising Pd on gamma alumina containing Grp IIB metal compounds for partial hydrogenation of acetylenic compounds. Bensalem et al, React. Kinet. Catal. Lett. Vol 60 No 1, 71-77 (1997) describe the reaction of Pd supported on ceria for the hydrogenation of but-1-yne.

As can be seen from considering the prior art in the field of acetylene hydrogenation, there is a need for an acetylene hydrogenation process and catalyst which is highly selective in order to maximise the conversion of acetylene in an olefin-containing feed, whilst being relatively inactive towards the olefinic bond.

According to the invention we provide a catalyst suitable for use in the hydrogenation of a hydrogenatable organic compound which comprises a palladium compound supported upon an alumina support material characterised in that said catalyst further comprises a promoter which comprises a compound of a lanthanide. The catalyst is particularly suitable for the hydrogenation of acetylenic compounds, especially for the selective hydrogenation of acetylenes in olefin-containing gas streams.

The catalyst is active for hydrogenation when the palladium is present in metallic form. The catalyst is usually made by first manufacturing a precursor in which a palladium compound, normally a salt or an oxide, is present on the support. It is normal commercial practice to supply such catalysts in the form of a reducible palladium compound supported upon an alumina support material, such that the reduction of the palladium compound to metallic palladium is carried out in situ in the reactor by the end-user of the catalyst. The term “catalyst” is used herein to refer both to the non-reduced form, in which the palladium is present In the form of a reducible palladium compound, and to the reduced form, in which the palladium is present as palladium metal. Thus the palladium compound may comprise a palladium salt, e.g. a nitrate or chloride, palladium oxide or palladium metal.

According to a second aspect of the invention, we further provide a process for the hydrogenation of a hydrogenatable organic compound comprising the step of passing a mixture of a gaseous feed containing said hydrogenatable organic compound and hydrogen over a catalyst comprising a palladium compound supported upon an alumina support material characterised in that said catalyst further comprises a promoter which comprises a compound of a lanthanide. The catalyst is especially suitable for the selective hydrogenation of acetylenic compounds, especially in the presence of other hydrogenateable compounds such as olefinic compounds. Thus the process of the invention in a preferred form comprises the selective hydrogenation of acetylene and/or higher alkynes in the presence of an olefin, e.g. ethylene.

The support may be selected from silica, titania, magnesia, alumina or other inorganic carriers such as calcium-aluminate cements. Preferably the support comprises alumina. A preferred alumina support material is predominantly an alpha-alumina. Alpha alumina is already well known for use as a support for palladium catalysts for use in hydrogenation reactions, as described for example in EP-A-0124744, U.S. Pat. No. 4,404,124, U.S. Pat. No. 3,068,303 and other references. It may be made by calcining an active alumina (e.g. gamma alumina or pseudoboehmite) at a temperature of 800-1400° C., more preferably 1000-1200° C. A detailed description of the effect on the physical properties of alumina of calcining at such temperatures is given in U.S. Pat. No. 3,113,980. Other forms of alumina may be used, for example active aluminas or transition aluminas as described in U.S. Pat. No. 4,126,645. Usually the support (for example an alpha-alumina) has a relatively low surface area. Following the teachings of the prior art, it is preferred that for use in “front-end” hydrogenation the surface area, as determined by the well known BET methodology is less than 50 m²g⁻¹ and more preferably less than 10 m²g⁻¹. The support is preferably of relatively low porosity, e.g. 0.05-0.5 cm³g⁻¹. Preferably the mean pore diameter lies within the range 0.05-1 micron, more preferably from about 0.05 to 0.5 microns.

The catalyst may be provided in any suitable physical form, but for fixed bed hydrogenation duty, shaped particles having a minimum dimension greater than 1 mm are preferred. The shaped particles may be in the form of cylinders, tablets, spheres or other shapes such as lobed cylinders, optionally with passages or holes. Alternatively, but less preferred are granules. Such particles may be formed by known methods such as tabletting, granulation, extrusion etc. Suitable particle dimensions are selected according to the conditions to be used, since the pressure drop through a bed of small particles is typically greater than through a bed of larger particles. Normally catalyst particles for hydrogenation of acetylene in refinery process streams have a minimum dimension of between about 2 and 5 mm, e.g. cylinders of about 3 mm diameter and 3 mm length are suitable. The catalyst support may be shaped into the desired particle form before the palladium and promoter compound is introduced or alternatively the supported catalyst may be shaped after manufacture. It is greatly preferred to use a preformed shaped catalyst support so that the application of palladium and promoter compound can be controlled to provide non-homogeneous catalyst particles if required. As mentioned previously, supported palladium catalysts are commonly supplied as shell-type catalysts in which the active metal is provided only at or near the surface of the catalyst. In order to achieve such a non-homogeneous distribution it is necessary to apply the active metals compounds after the support particle has been formed. Commercial catalyst supports are readily available in a variety of suitable particle shapes and sizes.

The palladium may be introduced into the catalyst by any suitable method as will be well known to the skilled catalyst manufacturer, e.g by impregnation of the support with a solution of a soluble palladium compound or by vapour deposition, as described in U.S. Pat. No. 5,063,194. A preferred method of manufacture is by impregnation of the support material with a solution of a soluble palladium salt such as palladium nitrate or palladium chloride, sulphate, acetate or of a palladium ammine complex. The incipient wetness technique is preferred, in which the volume of solution applied to the support is calculated to be sufficient to just fill the pores of the support material or to almost fill the pores e.g. the volume used may be about 90-95% of the calculated or measured pore volume. The concentration of the solution is adjusted to provide the required amount of palladium in the finished catalyst. The solution is preferably applied by spraying onto the support, normally at room temperature. Alternative methods such as dipping the support into the solution may be used. The impregnated support is then dried, and may then be subjected to treatment at elevated temperature to convert the impregnated palladium compound to an oxidic species. For example, when the palladium is applied to the support as a solution of palladium nitrate, the dried, impregnated material is preferably treated at a temperature above 400° C. in order to denitrify the material and form a more stable palladium species which is likely to be mainly palladium oxide.

The palladium is present at a level in the range of about 50 ppm to about 1% by weight based on Pd metal in the total catalyst weight, but the amount of palladium in the catalyst depends upon the intended use. For removal of acetylenic species from C₂ or C₃ gas streams, the palladium is present preferably at a level in the range of about 50 ppm to about 1000 ppm by weight, calculated on the weight of the total catalyst. More preferably the Pd level for this application is in the range 100-500 ppmw. When higher hydrocarbons are to be treated, e.g. in a pygas stream, then the catalyst typically includes a higher loading of palladium, e.g. 0.1% to 1%, more preferably about 0.2% -about 0.8%. The amount of Pd in a catalyst intended for “tail-end” duty may be greater than the amount required in a catalyst for “front-end” duty.

The lanthanide promoter compound may be Introduced into the catalyst by similar methods to those used for the palladium compound. That is, a solution of a soluble salt of the lanthanide compound may be impregnated into the support or sprayed onto the support. Suitable soluble compounds of the promoter include nitrates, basic nitrates, chlorides, acetates and sulphates. The palladium compound and the promoter compound may be introduced onto the support at the same time as each other or at separate times. For example a solution of the promoter compound may be applied to a formed material comprising a supported palladium compound. Alternatively a solution containing both a palladium compound and a lanthanide compound may be applied to the support material.

The promoter compound is a compound of a lanthanide, i.e. a compound of an element selected from La, Ce, Pr, Nd, Pm, Sm, Eu, Gd, Tb, Dy, Ho, Er, Tm, Yb and Lu. The preferred promoter compound is selected from; a compound of cerium, gadolinium or lanthanum, and is most preferably a cerium compound. The lanthanide compound is normally present in the catalyst in the form of an oxide, for example as Ce₂O₃ in the case of cerium.

The lanthanide promoter compound is present at a concentration of 15-8000 ppmw based on the promoter metal and the weight of the total catalyst, more preferably 50-5000 ppmw. When the promoter is a cerium compound the more preferred concentration is 50-2500 ppmw. In catalysts containing a higher concentration of Pd—e.g. for treatment of higher hydrocarbons such as pygas streams, the level of promoter may be increased up to e.g. 5% by weight. The atomic ratio of Pd to lanthanide promoter metal is preferably in the range 1:0.5-1:5, more preferably in the range 1:1 to 1:3.5.

It is preferred that the Pd and preferably also the lanthanide compound is present only in a layer at or near the surface of the support, i.e. that the catalyst is of the “shell” type. It is known that for use in selective hydrogenation, it is beneficial to use a catalyst in which the active component is concentrated in a relatively thin layer near the surface in order to minimise the contact time of the gas stream with the active catalyst and thereby increase selectivity. The active layer may be located beneath the surface of the support in order to improve its resistance to attrition. Typically In preferred catalysts the Pd and preferably also the lanthanide compound Is concentrated in a layer up to about 500 μm from the surface, especially between about 20 and 300 μm from the surface of the catalyst support.

A preferred embodiment of the catalyst of the present invention comprises an alumina catalyst support and a palladium compound and a promoter compound, said palladium compound being present at 50 ppmw-500 ppmw based on the weight of the catalyst, said promoter compound being selected from a compound of cerium, gadolinium or lanthanum, and being present at a concentration of 50-2500 ppmw based on the weight of the total catalyst.

The process and catalyst of the invention is useful to remove acetylene and higher acetylenes, for example methyl acetylene and vinyl acetylene from olefin streams.

Typical processes operate at pressures between 10 bar and 50 bar (gauge), especially up to about 20 bar. The temperature of operation depends upon the operating pressures but typically operate at inlet temperatures between 40 and 70° C. and outlet temperatures between 80 and 130° C., or higher, depending on the requirements of adjacent process steps in the plant.

The process and catalyst of the invention will be further described in the following examples.

Testing of the Catalysts (Front-End Conditions)

About 20 cm³ of whole catalyst pellets-(typically 20±1 cm³) was accurately weighed and then mixed with 315 g of an inert alumina diluent. The catalyst and diluent mixture was then charged to a tubular reactor having an internal diameter of 20 mm and a capacity of 200 cm³. The catalysts were pre-treated in-situ with 100% hydrogen at 20 bar, GHSV 5000 hr⁻¹, for at least 3 hours at 90° C., then purged with nitrogen whilst cooling to ambient temperature prior to commencing the test.

A model feed gas, designed to simulate de-ethaniser overhead front-end conditions was fed to the reactor at a gas hourly space velocity of 5,000 hr⁻¹ at a pressure of 20 bar gauge The composition of the gas feed was:

| | |
|---|---|
| Acetylene/mole % | 0.6 |
| Carbon monoxide/ppmv | 100 |
| Ethylene/mole % | 30.0 |
| Hydrogen/mole % | 15.0 |
| Nitrogen | balance |

The catalyst bed temperature was increased in ca. 2.5° C. steps to acetylene clean-up (T_{CUT}) which was taken to be achieved when the acetylene concentration in the exit gas was 3 ppmv or less. The experiment was continued by increasing the temperature by 1° C. steps until temperature runaway (T_{LOT}). As soon as an exotherm was detected, the reactors were quenched with process nitrogen to aid cooling and thereby flush out the potential reactants. All gas compositions were analysed by gas chromatography. By comparing the inlet and exit acetylene levels, acetylene conversion at a given temperature (Tₙ) was calculated from the following expression:
% C₂H_{2 Conv}=[(C₂H₂)ᵢₙ−(C₂H₂)ₒᵤₜ)/(C₂H₂))ᵢₙ]×100
where C₂H₂₀₍ᵢₙ₎ is the inlet level of acetylene, and, (C₂H₂)ₒᵤₜ is the outlet (exit) level of acetylene.

Ethylene selectivity (with respect to over-hydrogenation) was calculated by the following expression:
% S_{C2H4}=100−% S_{C2H6}
where % S_{C2H6} is the ethane selectivity as defined by the expression below:
% S_{C2H6}={[(C₂H₆)ₒᵤₜ−(C₂H₆)ᵢₙ]/[(C₂H₂)ᵢₙ−(C₂H₂)ₒᵤₜ[}×100

### EXAMPLE 1

A catalyst comprising 200 ppm Pd and the required amount of cerium to give a catalyst having a Pd:Ce atomic ratio between 1:0 and 1:10 was made by impregnating an alumina support, in the form of 3.2 mm diameter cylindrical pellets, by spraying at room temperature with a calculated volume of an aqueous solution of cerium (III) nitrate hexahydrate and palladium nitrate sufficient to fill the pores of the catalyst. The concentration of the cerium and palladium in the solution was adjusted to produce a catalyst having the required amount of each metal compound. This method of preparing a supported catalyst compound by the so-called “incipient wetness” method is well known to the skilled practitioner. The resulting material was dried at 105° C. in air for 3 hours and then heated to 450° C. in air for four hours to effect denitrification, i.e. to convert the cerium and palladium nitrates to oxidic species. The catalyst was tested under “front-end” conditions as described above and the results are shown in Table 1. The selectivity was calculated at the clean-up-temperature for each catalyst. The results show that compared with the unpromoted palladium catalyst, the LOT-CUT operability window is wider and the selectivity to ethylene is significantly better using the catalysts of the invention.

### EXAMPLE 2

A catalyst containing gadolinium instead of cerium was made by the method of Example 1 but substituting a solution of gadolinium nitrate (made using gadolinium (III) nitrate hexahydrate) for the cerium (III) nitrate hexahydrate. The Pd:Gd atomic ratio was 1:2. The catalyst was tested under “front-end” conditions as described above and the results are shown in Table 2.

### EXAMPLE 3

A catalyst containing lanthanum instead of cerium was made by the method of Example 1 but substituting a solution of lanthanum nitrate (made using lanthanum nitrate hexahydrate) for the cerium (III) nitrate hexahydrate. The Pd:La atomic ratio was 1:2. The catalyst was tested under “front-end” conditions as described above and the results are shown in Table 2.

TABLE 1

| | | Pd:Ce | | | LOT − | |
|---|---|---|---|---|---|---|
| | | atomic | CUT | LOT | CUT | % C₂H₄ |
| Catalyst | Promoter | ratio | (° C.) | (° C.) | (° C.) | selectivity |
| Comparison | None | — | 57 | 97 | 40 | 90.0 |
| Example 1a | Ce | 1:0.1 | 53 | 95 | 42 | 92.3 |
| Example 1b | Ce | 1:0.5 | 55 | 97 | 42 | 92.9 |
| Example 1c | Ce | 1:1 | 58 | 108 | 50 | 93.4 |
| Example 1d | Ce | 1:1.25 | 58 | 113 | 55 | 94.4 |
| Example 1e | Ce | 1:2 | 58 | 115 | 57 | 96.3 |
| Example 1f | Ce | 1:3 | 58 | 115 | 57 | 96.8 |
| Example 1g | Ce | 1:4 | 58 | 80 | 22 | 83.4 |
| Example 1h | Ce | 1:5 | 57 | 58 | 1 | 63.0 |

TABLE 2

| | | Pd:promoter | | | | |
|---|---|---|---|---|---|---|
| | | metal | | | LOT − | |
| | | (atomic | CUT | LOT | CUT | % C₂H₄ |
| Catalyst | Promoter | ratio) | (° C.) | (° C.) | (° C.) | selectivity |
| Com- | None | — | 57 | 97 | 40 | 90.0 |
| parison | | | | | | |
| Example 2 | Gd | 1:2 | 57 | 102 | 45 | 94.3 |
| Example 3 | La | 1:2 | 58 | 110 | 52 | 95.1 |

### EXAMPLE 4

Two catalysts were made containing 400 ppm of Pd. One (designated 4a) was unpromoted and the other (4b) contained cerium at an atomic ratio of Pd:Ce of 1:2. The catalysts were prepared by impregnation of the alumina support with aqueous solutions of palladium (and cerium, if present) nitrates according to the general procedure described in Example 1. The catalysts were tested under tail-end hydrogenation conditions as described below.

Testing of the Catalysts (Tail-End Conditions)

20 cm³ of whole catalyst pellets were mixed with 315 g of inert alumina diluent and charged to a tubular reactor. The catalysts were pre-treated in-situ with 100% hydrogen at 20 bar, GHSV 5000 hr⁻¹, for at least 3 hours at 90° C., then purged with nitrogen whilst cooling to ambient temperature prior to commencing the test. A model feed gas, designed to simulate tail-end conditions, was fed to the reactor at a gas hourly space velocity of 2000 hr⁻¹ at a pressure of 17 bar gauge. The composition of the gas feed was:

| | |
|---|---|
| Acetylene/mole % | 1.00 |
| Hydrogen/mole % | 1.05 |
| Ethylene/mole % | balance |

The catalyst bed temperature was increased in steps of 5° C. to acetylene clean-up temperature (T_{CUT}) which was taken to be achieved when the acetylene concentration in the exit gas was 3 ppmv or less. All gas compositions were analysed by gas chromatography. By comparing the inlet and exit acetylene levels, acetylene conversion at a given temperature (Tₙ), and ethylene selectivity were calculated using the method and equations given for the front-end tests described above. Total butene make (the sum of 1-butene, cis-2-butene and trans-2-butene) and also 1,3-butadiene make at the clean-up temperature was calculated as follows:
Butene make (ppmv)=(total butene)ₒᵤₜ−(total butane)ᵢₙ (ppmv)

and similarly for 1,3-butadiene make:
Butadiene make (ppmv)=(butadiene)ₒᵤₜ−(butadiene)ᵢₙ (ppmv).

The results are shown in Table 3 and show a significant improvement in ethylene selectivity when the Ce-promoted catalyst is used. In addition to lower levels of ethane due to over-hydrogenation, the levels of C₄ compounds (butadiene and butenes) are significantly reduced. These materials are not present in the feed gas and are formed by oligomerisation of the C₂ compounds. They are thought to be the precursors to the “green oils” that cause catalyst deactivation.

TABLE 3

| | Pd:Ce | | Ethane | Butenes | Butadiene | C₂H₄ |
|---|---|---|---|---|---|---|
| | (atomic | CUT | make | make | make | selectivity |
| Catalyst | ratio) | (° C.) | (ppm) | (ppm) | (ppm) | (%) |
| 4a | 1:0 | 38 | 217 | 45 | 3466 | 97.8 |
| (comparison) | | | | | | |
| 4b | 1:2 | 43 | 107 | 93 | 346 | 99.2 |

1. A catalyst suitable for use in the hydrogenation of a hydrogenatable organic compound which consists essentially of a palladium compound supported upon a support material characterised in that said catalyst further comprises a compound of a lanthanide.

2. A catalyst as claimed in claim 1 wherein the support is selected from silica, titania, magnesia, alumina, silica-alumina, a calcium-aluminate cement or a mixture of these compounds.

3. A catalyst as claimed in claim 2 wherein the support comprises alumina.

4. A catalyst as claimed in any of claims 1-3 wherein the mean pore diameter lies within the range 0.05-1 micron.

5. A catalyst as claimed in any of the preceding claims wherein the catalyst is in the form of shaped particles having a minimum dimension greater than 1 mm.

6. A catalyst as claimed in any of the preceding claims, wherein the lanthanide compound is a compound of cerium, gadolinium or lanthanum.

7. A catalyst as claimed in claim 6, wherein the lanthanide compound is a compound of cerium

8. A catalyst as claimed in any one of the preceding claims wherein the palladium is present at a level in the range of about 50 ppm to about 1% by weight calculated as Pd metal and the weight of the total catalyst.

9. A catalyst as claimed in any of the preceding claims wherein the lanthanide compound is present at a concentration of 50-5000 ppmw based on the lanthanide metal and the weight of the total catalyst.

10. A catalyst as claimed in any of the preceding claims wherein the atomic ratio of Pd to lanthanide metal is in the range 1:0.5-1:3.5.

11. A catalyst as claimed in any of the preceding claims wherein the palladium is present in the form of palladium metal.

12. A process for the hydrogenation of a hydrogenatable organic compound comprising the step of passing a mixture of a gaseous feed containing said hydrogenatable organic compound and hydrogen over a catalyst which consists essentially of a palladium compound supported upon a support material characterised in that said catalyst further comprises a compound of a lanthanide.

13. A hydrogenation process as claimed in claim 12, wherein said hydrogenatable organic compound comprises an acetylenic compound.

14. A process as claimed in claim 13, wherein said gaseous feed stream contains a minor proportion of an acetylenic compound and a major proportion of an olefinic compound, in addition to hydrogen.

15. A process as claimed in claim 13 or claim 14, wherein said gaseous feed stream contains a minor proportion of acetylene and a major proportion of ethylene, in addition to hydrogen.

16. A process as claimed in any one of claims 12 to 15, wherein said catalyst is a catalyst as claimed in any one of claims 1-11.
